(19) United States
(12) Patent Application Publication
(10) Pub. No.: US 2022/0327164 A1
(43) Pub. Date: Oct. 13, 2022

(54) DATA PROCESSING ASSISTANT SYSTEM, DATA PROCESSING ASSISTANT METHOD, AND DATA PROCESSING ASSISTANT PROGRAM

(71) Applicant: Hitachi, Ltd., Tokyo (JP)

(72) Inventors: Mika Takata, Tokyo (JP); Norifumi Nishikawa, Tokyo (JP); Rikiya Tajiri, Tokyo (JP); Yusuke Funaya, Tokyo (JP); Toshihiko Kashiyama, Tokyo (JP)

(73) Assignee: Hitachi, Ltd., Tokyo (JP)

(21) Appl. No.: 17/642,373

(22) PCT Filed: Mar. 11, 2021
(86) PCT No.: PCT/JP2021/009790

§ 371 Date: Mar. 11, 2022

(30) Foreign Application Priority Data
Mar. 25, 2020 (JP) 2020-053983

(51) Int. Cl. G06F 16/9032; G06F 16/906; G06F 16/901

(52) U.S. Cl. CPC G06F 16/90332; G06F 16/906; G06F 16/9027

(57) ABSTRACT

Provided is a data processing assistant system, a data processing assistant method, and a data processing assistant program. The data processing assistant system includes: a processing record accumulation unit that accumulates processing records in which one or more pieces of data, data processing performed using the data, and a processing result of the data processing are associated with each other; a correspondence relation data creation unit that creates, based on the processing records, correspondence relation data indicative of a correspondence relation among a data type indicating a type of the data, a question to be solved by the data processing, and the processing result; and a processing information presentation unit that presents, upon receiving designation of the data type and the question, information related to appropriate data processing based on the correspondence relation data.

### BACKGROUND OF THE INVENTION

### 1. Field of the Invention

The present invention relates to a data processing assistant system, a data processing assistant method, and a data processing assistant program that assists data processing.

### 2. Description of the Related Art

In the related art, JP-A-2019-185751 discloses a technique of assisting data processing. This publication describes “receiving patient feature data; determining similarity of pre-stored models with the patient feature data, wherein a database of the pre-stored models is analyzed to assess similarity indicating that feature preparation of the pre-stored models is compatible with the patient feature data; for similarity indicative of feature preparation to be utilized: conducting the feature preparation for the patient feature data based on the pre-stored model determined to be similar, wherein the feature preparation retrieves reusable features associate with the pre-stored model determined to be similar, where the reusable features comprise pre-calculated features of the pre-stored model determined to be similar; generating a machine learning model using results of the feature preparation and patient feature data; and providing a prediction using the machine learning model”.

According to JP-A-2019-185751, it is possible to quickly conduct model preparation by reusing the features and the like. However, the model preparation requires specialized knowledge, and thus it is still difficult for general users (users without advanced skills) to use. Therefore, for example, it is required to assist the use of data processing even for the general users by presenting analyzable content, necessary data, prediction accuracy, etc. based on past analysis.

### SUMMARY OF THE INVENTION

An object of the invention is to assist in data processing by providing a variety of information pertaining to data processing.

In order to achieve the above object, one of the representative data processing assistant system, the data processing assistant method, and the data processing assistant program of the invention accumulates processing records in which one or more pieces of data, data processing performed using the data, and a processing result of the data processing are associated with each other; creates, based on the processing records, correspondence relation data indicative of a correspondence relation among a data type indicating a type of the data, a question to be solved by the data processing, and a processing result; and presents, upon receiving designation of the data type and the question, information related to appropriate data processing based on the correspondence relation data.

According to the invention, data processing can be assisted by providing a variety of information pertaining to the data processing. Problems, configurations, and effects other than those described above will be clarified by the following description of an embodiment.

### BRIEF DESCRIPTION OF THE DRAWINGS

FIG. 1 is a schematic diagram of data processing assistant according to an embodiment;

FIG. 2 is a schematic diagram of a hierarchical structure of correspondence structure data;

FIG. 3 is a schematic diagram of a specific example of correspondence structure data;

FIG. 4 is a system configuration diagram of a data processing assistant system;

FIG. 5 is a flowchart showing correspondence structure data creating processing;

FIG. 6 is a flowchart showing a processing operation related to information presentation;

FIG. 7 is a flowchart showing details of processing information presentation processing;

FIG. 8 is a flowchart showing details of similarity calculation processing;

FIG. 9 is a flowchart showing details of question searching processing;

FIG. 10 is a flowchart showing details of necessary data type searching processing;

FIG. 11 shows a specific example of data processing management data (1);

FIG. 12 shows a specific example of data processing management data (2);

FIG. 13 shows a specific example of data processing management data (3);

FIG. 14 shows a specific example of an adaptation table;

FIG. 15 shows a specific example of an alternative table;

FIG. 16 shows a specific example of screen display (1);

FIG. 17 shows a specific example of screen display (2); and

FIG. 18 shows a specific example of screen display (3).

### DESCRIPTION OF THE PREFERRED EMBODIMENTS

Hereinafter, an embodiment will be described with reference to the drawings.

### EMBODIMENT

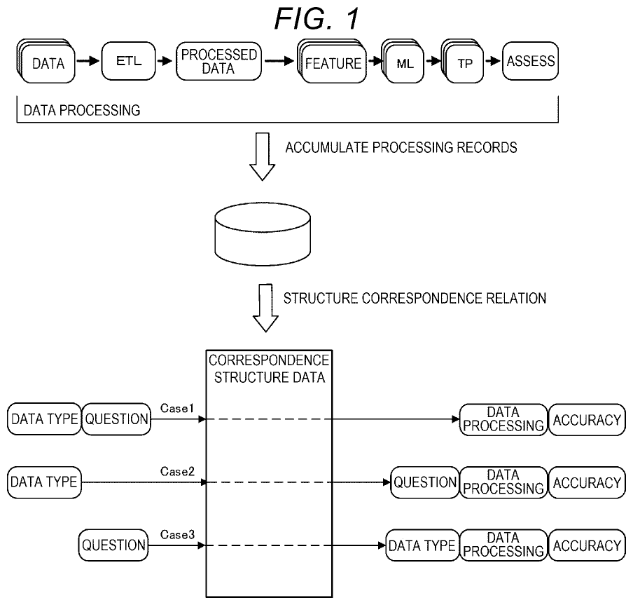

FIG. 1 is a schematic diagram of data processing assistant according to an embodiment. A data processing assistant system accumulates processing records of data processing and creates correspondence structure data based on the accumulated processing records. The data processing is a series of processing from working on one or more pieces of data, generating a feature from the processed data, inputting the feature to a machine learning model, and setting output of the machine learning model as a processing result. When working on one or more pieces of data, extract-transform-load (ETL) processing and the like can be used. The machine learning model is a combination of machine learning (ML) and tuning parameters (TP). It is also possible to assess the processing result and feed the processing result back to the machine learning model.

As a specific example of the data processing, there is processing of receiving blood pressure and medication history as data and calculating a readmission rate after a predetermined period. In the data processing, various processing such as working on data and input to the machine learning model is performed, and the data processing assistant system handles, as one data processing, processing of outputting the processing result (readmission rate or the like) as a final ending point from data (blood pressure or the like) as a starting point given at the beginning of the series of processing. A type of data as the starting point is referred to as data type, and an item to be solved by the data processing is referred to as a question. That is, “blood pressure” is the data type, and “readmission rate after a predetermined period” is a question to be solved by the data processing. The processing result of the data processing in which “readmission rate after a predetermined period” is the question is represented by a probability such as “30%”. As an assessment of the processing result, prediction accuracy (accuracy, AUC and the like) and various statistical indices (f-measure, precision, recall, and the like) can also be calculated. For example, when the processing result of “readmission rate after a predetermined period” is “30%” and the prediction accuracy thereof is “80%”, the prediction of “a target person readmits at a probability of 30%” “hits at a probability of 80%.”

The data processing assistant system accumulates a large number of processing records of the data processing and creates correspondence structure data by structuring the correspondence relation between the data type, the question, and the processing result. Details will be described later, and the correspondence structure data has a hierarchical structure including a question layer, a data type layer, and a processing record layer. This correspondence structure data corresponds to the correspondence relation data described in the claims.

When receiving designation of the data type and the question (case 1), the data processing assistant system can present information related to appropriate data processing based on the correspondence structure data. Specifically, the data processing assistant system can specify data processing applicable to a designated data type and a designated question and present expected accuracy to the processing result.

Further, when receiving designation of the data type (case 2), the data processing assistant system can output an answerable question, applicable data processing, and expected accuracy to the processing result with reference to the correspondence structure data.

Similarly, when receiving designation of the question (case 3), the data processing assistant system can output a data type necessary for an answer, applicable data processing, and expected accuracy to the processing result with reference to the correspondence structure data.

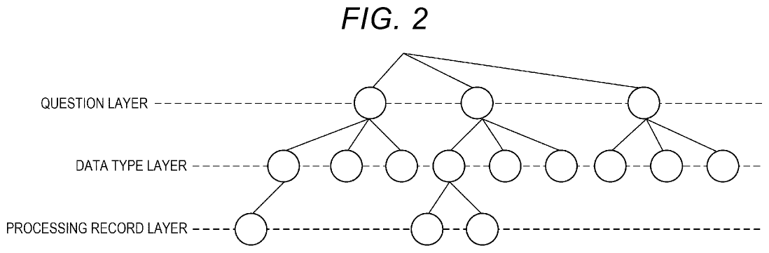

FIG. 2 is a schematic diagram of a hierarchical structure of the correspondence structure data. As shown in FIG. 2, the correspondence structure data has a hierarchical structure including a question layer having a node indicating the question, a data type layer having a node indicating the data type, and a processing record layer having a node indicating the processing record.

Each node is connected to a single upper node when connected to an upper node located in a relatively upper layer, and is connected to one or more lower nodes when connected to a lower node located in a relatively lower layer. Therefore, the correspondence structure data has a tree structure. An order of layers is the question layer, the data type layer, and the processing record layer from the top. Further, there may be another layer above the question layer. There may be a plurality of question layers and data type layers.

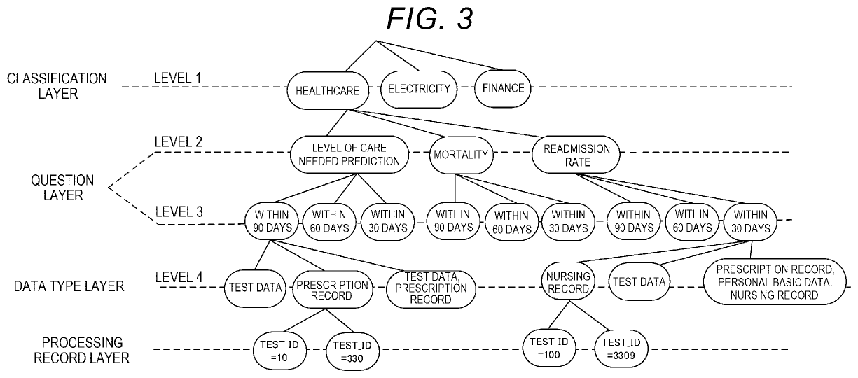

FIG. 3 is a schematic diagram of a specific example of the correspondence structure data. The correspondence structure data shown in FIG. 3 includes a classification layer higher than the question layer, two question layers, one data type layer, and one processing record layer. The classification layer indicates a classification to which a question belongs. A lower question layer shows details of an upper question layer. Here, for convenience, layers until immediately before the processing record layer are called an input layer, and “number of level” is added from the top. Therefore, the classification layer is level 1 of the input layer, the question layers are level 2 and level 3 of the input layer, and the data type layer is level 4 of the input layer.

The correspondence structure data shown in FIG. 3 includes “healthcare”, “electricity”, and “finance” nodes in the classification layer that is level 1. The “healthcare” node connects to three nodes in the question layer that is level 2. Specifically, the three nodes are “level of care needed prediction”, “mortality”, and “readmission rate”.

Further, each node in the question layer that is level 2 is connected to nodes in the question layer that is level 3. Specifically, each node at level 2 is connected to three nodes of “within 90 days”, “within 60 days”, and “within 30 days”. The nodes at level 3 detail the nodes at level 2, and the nodes are treated individually even though names thereof are the same. The node of “within 60 days” connected to “level of care needed prediction” indicates “level of care needed prediction within 60 days”, and the node of “within 60 days” connected to “mortality” indicates “mortality rate within 60 days”.

The number and contents of the nodes at level 3 can be set individually according to the nodes at level 2. For example, when the node at level 2 is “survival rate for cancer”, it is desirable to have a yearly node at level 3.

Nodes in the data type layer are types of data as the starting point for the data processing. Here, an individual node is provided for a combination of a plurality of data types. In FIG. 3, “test data”, “prescription record”, and “test data, prescription record” are connected as the nodes connected to “level of care needed prediction within 90 days”. Similarly, “nursing record”, “test data”, and “prescription record, personal basic data, nursing record” are connected as the nodes connected to “readmission rate within 30 days”.

A node in the processing record layer corresponds to an actual processing result. In FIG. 3, “TEST_ID=10” and “TEST_ID=330” are connected to the node of the prescription record, and are each identification information attached to a processing result of one corresponding data processing.

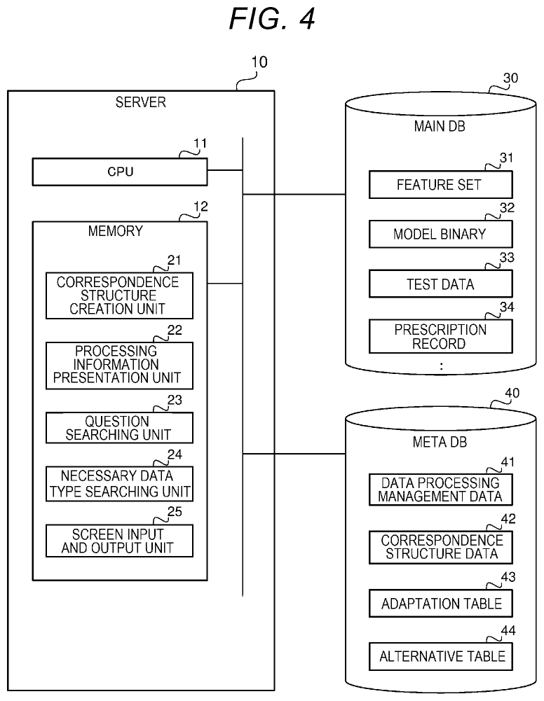

Next, a system configuration of the data processing assistant system will be described. FIG. 4 is a system configuration diagram of the data processing assistant system. As shown in FIG. 4, the data processing assistant system includes a server 10, a main database (DB), and a meta DB 40.

The server 10 includes a central processing unit (CPU) and a memory 12. The CPU 11 operates as various functional units by loading and executing a program read from an auxiliary storage device (not shown) on the memory 12 that is a main storage device. FIG. 4 shows a state in which a program operating as a correspondence structure creation unit 21, a processing information presentation unit 22, a question searching unit 23, a necessary data type searching unit 24, and a screen input and output unit 25 is loaded in the memory 12.

A main DB 30 is a database that stores data as the starting point for data processing in addition to a feature set 31 and a model binary 32. The data as the starting point of the data processing includes test data 33, a prescription record 34, and the like. The feature set 31 is a data group worked on for input to the machine learning model. The model binary 32 is data that identifies the machine learning model.

The meta DB 40 is a database that stores data processing management data 41, correspondence structure data 42, an adaptation table 43, an alternative table 44, and the like. The data processing management data 41 is data in which the processing records of the data processing are accumulated. The correspondence structure data 42 is data that uniquely specifies the correspondence structure. The adaptation table 43 is a data table for registering data processing performed under the same condition as the designated data type and the designated question. The alternative table 44 is a data table for registering data processing performed under a similar condition as the designated data type and the designated question.

Based on the processing records, the correspondence structure creation unit 21 creates the correspondence structure data 42 indicative of a correspondence relation among a data type indicating a type of the data, a question to be solved by the data processing, and a processing result, and stores the correspondence structure data 42 in the meta DB 40.

When receiving the designation of the data type and the question, the processing information presentation unit presents information related to appropriate data processing based on the correspondence structure data 42. Specifically, when tracing the hierarchical structure of the correspondence structure data 42 based on the designated data type and the designated question from the upper level and reaching the node connected to the processing record layer (a node in the lowest input layer), the processing information presentation unit 22 registers the data processing related to the processing records connected to the node in the adaptation table 43, and presents the data processing of the adaptation and the accuracy of the answer by the adaptation. The processing information presentation unit 22 obtains similarity between the designated data type and the designated question and a route by which the hierarchical structure is traced from the upper level, registers the data processing related to the processing records connected to the route having strong similarity in the alternative table 44, and presents the data processing of the alternative and the accuracy of the answer by the alternative.

When receiving the designation of the data type, the question searching unit 23 selects a node having a high matching level from the node in the data type layer and outputs the node in the question layer on a route to the node having a high matching level as an answerable question candidate. Thereafter, the processing information presentation unit 22 can present information related to appropriate data processing using the designated data type and the question candidate.

When receiving the designation of the question, the necessary data type searching unit 24 traces the hierarchical structure of the correspondence structure data 42 from the upper level based on the designated question, and outputs the node in the data type layer located below the node where the necessary data type searching unit 24 reaches as the necessary data type. The processing information presentation unit 22 can present information related to appropriate data processing using the designated data type and the necessary data type.

The screen input and output unit 25 performs output control of a display screen on a display unit (not shown) connected to the server 10, and input reception according to the display screen. In addition, although not shown, the data processing assistant system includes a database management system (DBMS) for the main DB 30 and a DBMS for the meta DB 40.

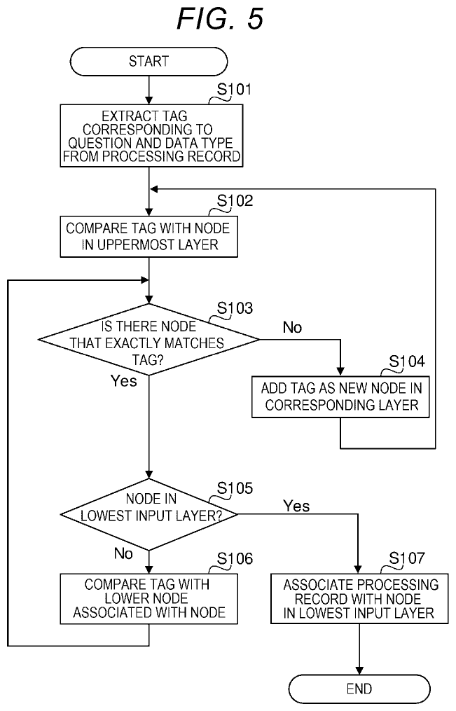

FIG. 5 is a flowchart showing correspondence structure data creating processing. The flowchart of FIG. 5 includes the following steps.

### (Step S101)

In a processing start step, the correspondence structure creation unit 21 extracts a tag corresponding to the question and the data type from the processing records related to one data processing, and proceeds to step S102.

### (Step S102)

The correspondence structure creation unit 21 compares the tag with the node in the uppermost layer of the correspondence structure data 42, and proceeds to step S103.

### (Step S103)

If there is no node that exactly matches the tag (step S103; No), the correspondence structure creation unit 21 proceeds to step S104. If there is a node that exactly matches the tag (step S103; Yes), the correspondence structure creation unit 21 proceeds to step S105.

### (Step S104)

The correspondence structure creation unit 21 adds the tag corresponding to the uppermost layer as a new node in the layer, and proceeds to step S102.

### (Step S105)

The correspondence structure creation unit 21 determines whether the node that exactly matches the tag is the node in the lowest input layer. If the node is not the node in the lowest input layer (step S105; No), the correspondence structure creation unit 21 proceeds to step S106. If the node is the node in the lowest input layer (step S105; Yes), the correspondence structure creation unit 21 proceeds to step S107.

### (Step S106)

The correspondence structure creation unit 21 compares the tag with a lower node associated with the node, and proceeds to step S103.

### (Step S107)

The correspondence structure creation unit 21 associates the processing record with the node in the lowest input layer and ends the processing.

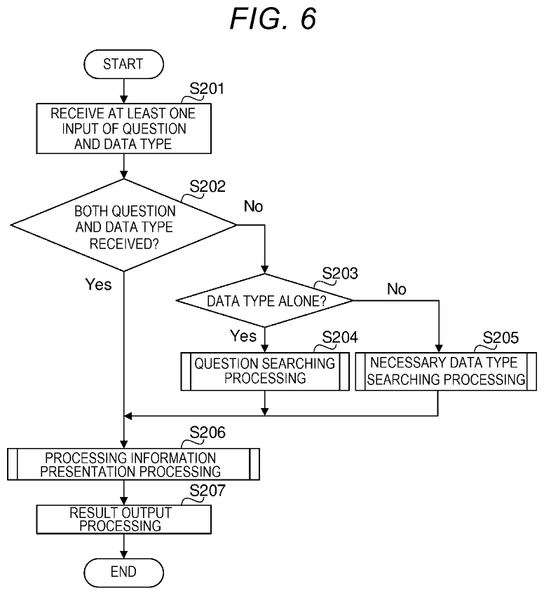

FIG. 6 is a flowchart showing a processing operation related to information presentation. The flowchart of FIG. 6 includes the following steps.

### (Step S201)

In a processing start step, the screen input and output unit 25 receives at least one of the question and the data type, and proceeds to step S202.

### (Step S202)

The processing information presentation unit 22 determines whether both the question and the data type are received. If both are received (step S202; Yes), the processing information presentation unit 22 proceeds to step S206. If only one of the question and the data type is received (step S202; No), the processing information presentation unit 22 proceeds to step S203.

### (Step S203)

The processing information presentation unit 22 determines whether only the data type is received. If only the data type is received (step S203; Yes), the processing information presentation unit 22 proceeds to step S204. When the data type is not received (step S203; No), that is, when the problem is received, the processing information presentation unit 22 proceeds to step S205.

### (Step S204)

The question searching unit 23 executes the question searching processing, and proceeds to step S206. The details of the question searching processing will be described later.

### (Step S205)

The necessary data type searching unit 24 executes the necessary data type searching processing, and proceeds to step S206. The details of the necessary data type searching processing will be described later.

### (Step S206)

The processing information presentation unit 22 executes the processing information presentation processing, and proceeds to step S207. The details of the processing information presentation processing will be described later, and in this processing, the adaptation and the alternative are registered in the table.

### (Step S207)

The screen input and output unit 25 displays the adaptation and the alternative on the screen, and ends the processing. The adaptation may be read from the adaptation table 43. Similarly, the alternative may be read from the alternative table 44.

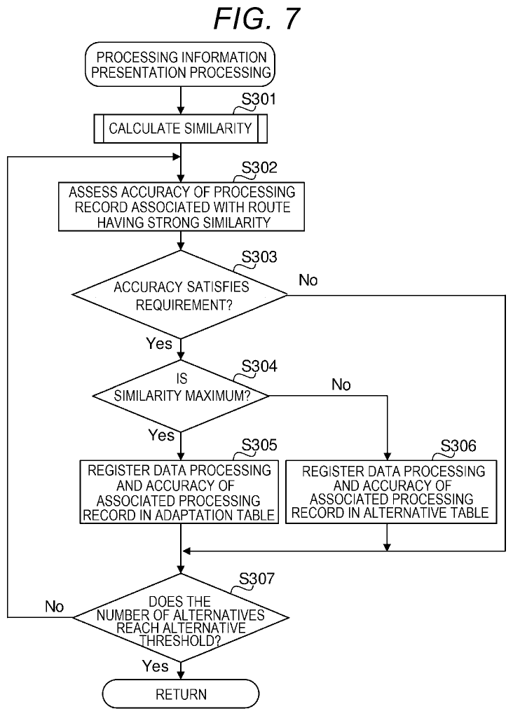

FIG. 7 is a flowchart showing details of the processing information presentation processing shown in FIG. 6. The flowchart of FIG. 7 includes the following steps.

### (Step S301)

In a processing start step, the processing information presentation unit 22 performs similarity calculation processing for calculating the similarity between the designated data type and the designated question and the route by which the hierarchical structure is traced from the upper level, and proceeds to step S302. The details will be described later, and the similarity is a maximum value in the route in which the designated data type matches with the designated question. In other words, the route having the maximum similarity indicates that there is a processing record for the same data type and the same question as the designated data type and the designated question.

### (Step S302)

The processing information presentation unit 22 assesses the accuracy of the processing record associated with the route having strong similarity, and proceeds to step S303.

### (Step S303)

The processing information presentation unit 22 determines whether the accuracy of the processing record associated with the route having strong similarity satisfies a requirement. If the requirement is not satisfied (step S303; No), the processing information presentation unit 22 proceeds to step S307. If the requirement is satisfied (step S303; Yes), the processing information presentation unit 22 proceeds to step S304.

### (Step S304)

The processing information presentation unit 22 determines whether the similarity is maximum. If the similarity is maximum (step S304; Yes), the processing information presentation unit 22 proceeds to step S305. If the similarity is not maximum (step S304; No), the processing information presentation unit 22 proceeds to step S306.

### (Step S305)

The processing information presentation unit 22 registers the data processing and accuracy of the processing record associated with the route having the maximum similarity as the adaptation in the adaptation table 43, and proceeds to step S307.

### (Step S306)

The processing information presentation unit 22 registers the data processing and accuracy of the processing record associated with the route having similarity that is not maximum as the alternative in the alternative table 44, and proceeds to step S307.

### (Step S307)

The processing information presentation unit 22 determines whether the number of the alternatives reaches an alternative threshold. If the number of the alternatives does not reach the alternative threshold (step S307; No), the processing information presentation unit 22 proceeds to step S302. If the number of the alternatives reaches the alternative threshold (step S307; Yes), the processing information presentation unit 22 returns to original processing.

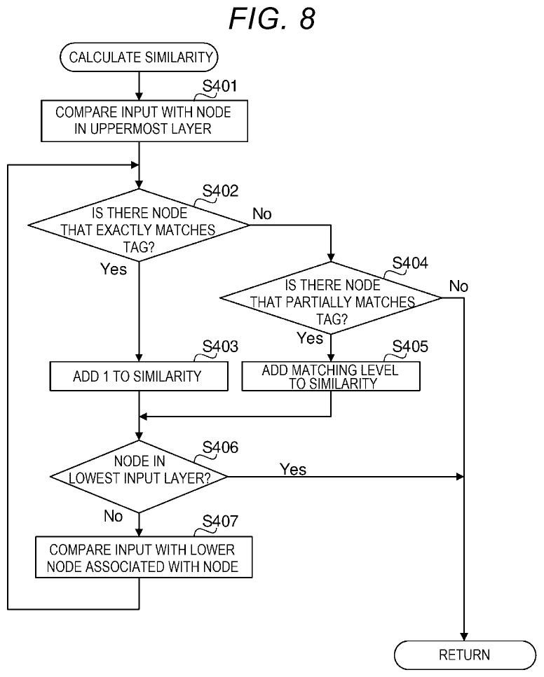

FIG. 8 is a flowchart showing details of the similarity calculation processing shown in FIG. 7. The flowchart of FIG. 8 includes the following steps.

### (Step S401)

In a processing start step, the processing information presentation unit 22 compares the input with the node in the uppermost layer, and proceeds to step S402.

### (Step S402)

If there is a node that exactly matches the input (step S402; Yes), the processing information presentation unit 22 proceeds to step S403. If there is no node that exactly matches the input (step S402; Yes), the processing information presentation unit 22 proceeds to step S404.

### (Step S403)

The processing information presentation unit 22 adds 1 to the similarity and proceeds to step S406.

### (Step S404)

If there is the node that partially matches the input (step S404; Yes), the processing information presentation unit 22 proceeds to step S405. If there is no node that partially matches the input (step S404; No), the processing information presentation unit 22 ends the similarity calculation processing and returns to the original processing. Here, the exact match and the partial match will be described. When there is a node (A, B) in the data type layer and (A, B) is given as the input, the input exactly matches with the node. On the other hand, when there is the node (A, B) in the data type layer and (B) is given as an input, the input exactly matches with the node.

### (Step S405)

The processing information presentation unit 22 adds a matching level to the similarity and proceeds to step S406. The matching level may be calculated by, for example, Dice Index.

### (Step S406)

The processing information presentation unit 22 determines whether the compared node is a node located in the lowest input layer. If the node is the node located in the lowest layer (step S406; Yes), the processing information presentation unit 22 ends the similarity calculation processing and returns to the original processing. If the node is not the node located in the lowest layer (step S406; No), the processing information presentation unit 22 proceeds to step S407.

### (Step S407)

The processing information presentation unit 22 compares the input with the lower node associated with the compared node, and proceeds to step S402 to trace the node to the lower layer.

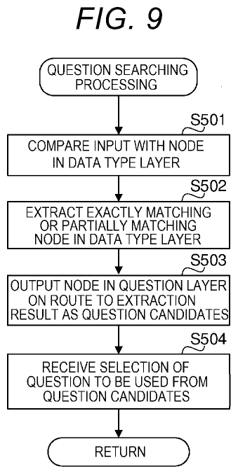

FIG. 9 is a flowchart showing details of the question searching processing shown in FIG. 6. The flowchart of FIG. 9 includes the following steps.

### (Step S501)

In a processing start step, the question searching unit 23 compares the input with the node in the data type layer, and proceeds to step S502.

### (Step S502)

The question searching unit 23 extracts an exactly matching or partially matching node in the data type layer, that is, a node having a high matching level, and proceeds to step S503.

### (Step S503)

The question searching unit 23 outputs nodes in the question layer on the route to the node of the extraction result as answerable question candidates, and proceeds to step S504.

### (Step S504)

The screen input and output unit 25 displays and outputs the question candidates, receives selection input of the question to be used from the question candidates, ends the question searching processing, and returns to the original processing. Thereafter, the processing information presentation unit 22 performs the processing information presentation processing (step S206) using the question selected in the question searching processing and the data type input in advance.

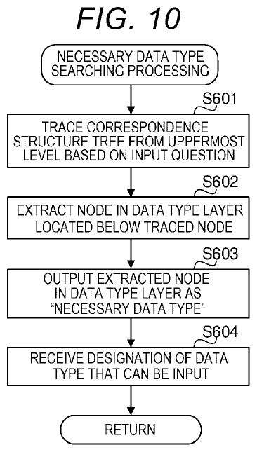

FIG. 10 is a flowchart showing details of the necessary data type searching processing shown in FIG. 6. The flowchart of FIG. 10 includes the following steps.

### (Step S601)

In a processing start step, the necessary data type searching unit 24 traces the hierarchical structure of the correspondence structure data 42 from the upper level based on the input question, and proceeds to step S602.

### (Step S602)

The necessary data type searching unit 24 extracts the node in the data type layer located below the traced node, and proceeds to step S603.

### (Step S603)

The necessary data type searching unit 24 outputs the extracted node in the data type layer as the necessary data type, and proceeds to step S604.

### (Step S604)

The screen input and output unit 25 displays and outputs the necessary data type, receives the designation of the data type that can be input, ends the necessary data type searching processing, and returns to the original processing. Thereafter, the processing information presentation unit 22 performs the processing information presentation processing (step S206) using the necessary data type for the designated data type in the necessary data type searching processing and the question input in advance.

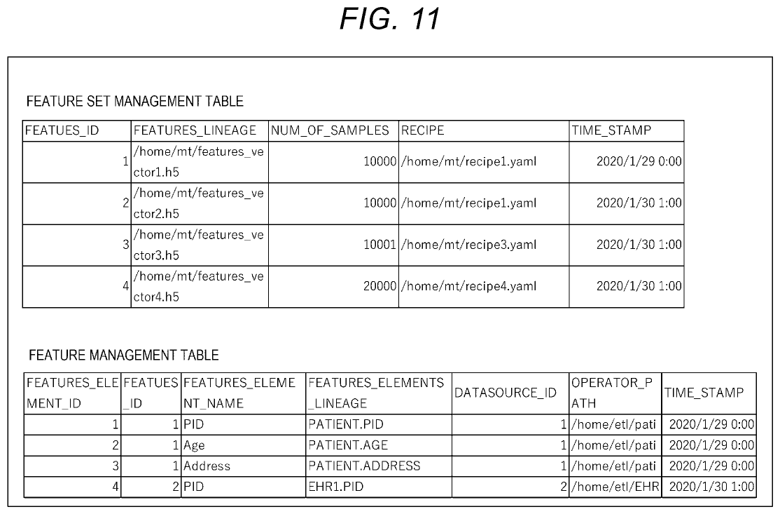

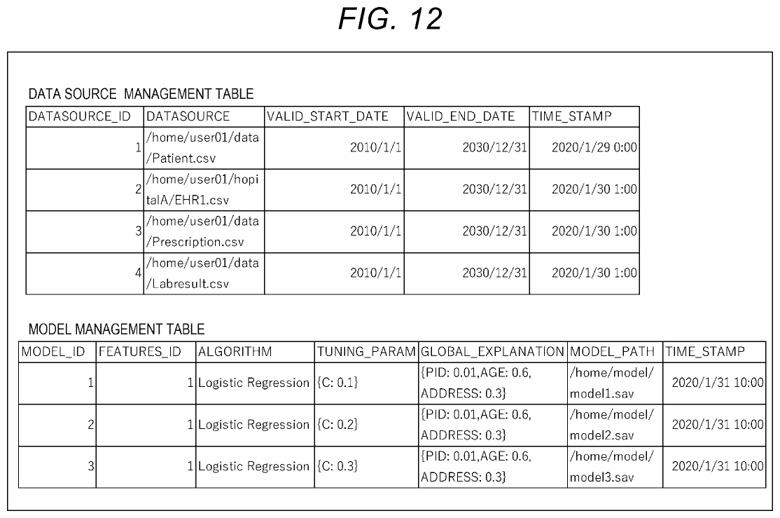

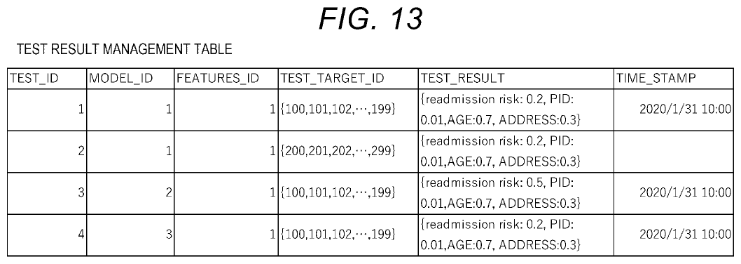

FIGS. 11 to 13 are specific examples of the data processing management data 41. As shown in FIGS. 11 to 13, the data processing management data 41 has a feature set management table, a feature management table, a data resource management table, a model management table, and a test result management table. These tables are linked to each other by an item “****_ID”.

The feature set management table has items of “FEATURES_ID”, “FEATURES_LINEAGE”, “NUM_OF_SAMPLES”, “RECIPE”, and “TIME_STAMP”, and manages a storage destination, a generation method, and generation date and time of each feature data.

The feature management table has items of “FEATURES_ELEMENT_ID”, “FEATURES_ID”, “FEATURES_ELEMENT_NAME”, “FEATURES_ELEMENTS_LINEAGE”, “DATASOURCE_ID”, “OPERATOR_PATH”, and “TIME_STAMP”, and manages a feature element name, a storage destination, a data source, generation date and time, etc.

The data resource management table has items of “DATASOURCE_ID”, “DATASOURCE”, “VALID_START_DATE”, “VALID_END_DATE”, and “TIME_STAMP”, and manages a validity period and generation date and time of each data source. Similarly, the model management table has items of “MODEL_ID”, “FEATURES_ID”, “ALGORITHM”, “TUNING_PARAM”, “GLOBAL_EXPLANATION”, “MODEL_PATH”, and “TIME_STAMP” to manage a model. The test result management table has items of “TEST_ID”, “MODEL_ID”, “FEATURES_ID”, “TEST_TARGET_ID”, “TEST_RESULT”, and “TIME_STAMP” to manage test results (processing results).

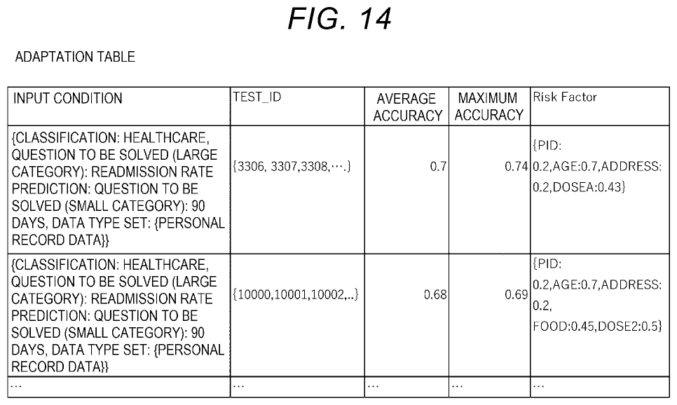

FIG. 14 is a specific example of the adaptation table 43. As shown in FIG. 14, the adaptation table 43 has items of “input condition”, “TEST_ID”, “average accuracy”, “maximum accuracy”, and “Risk Factor”, and manages the adaptation.

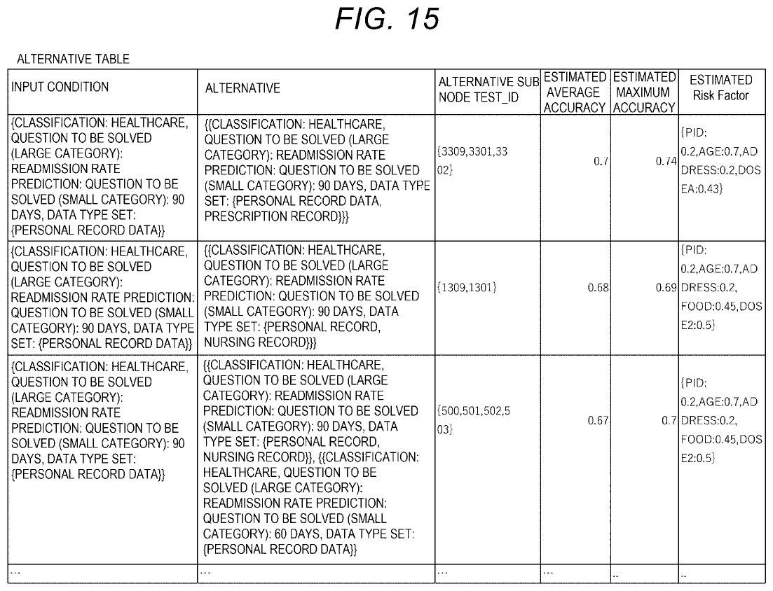

FIG. 15 is a specific example of the alternative table 44. As shown in FIG. 15, the adaptation table 44 has items of “input condition”, “alternative”, “alternative sub node TEST_ID”, “estimated average accuracy”, “estimated maximum accuracy”, and “estimated Risk Factor”, and manages the alternative.

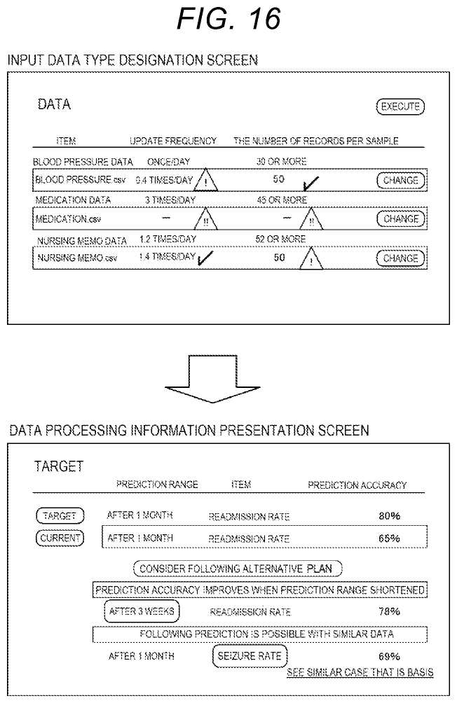

FIGS. 16 to 18 are specific examples of screen display by the screen input and output unit 25. On an input data type designation screen of FIG. 16, blood pressure data, medication data, and nursing memo data are designated as data types to be input. Here, in order to perform data processing with good accuracy, target values for an update frequency, the number of records per sample, etc. are set in the input items. Further, items that do not meet the target value are displayed with a warning.

On the data processing information presentation screen of FIG. 16, a prediction range, a question item, and prediction accuracy are displayed. Specifically, the data processing information presentation screen shows that the readmission rate after 1 month can be predicted with an accuracy of 65% when the designated data type is used. However, the target of prediction accuracy is 80%, and the prediction accuracy does not meet the target. Thus, as the alternatives, “the prediction accuracy improves when the prediction range shortened” and “predict other questions with similar data” are mentioned.

The alternative of “the prediction accuracy improves when the prediction range shortened” shows that the readmission rate can be predicted with an accuracy of 78% by changing the prediction range into after 3 weeks. Similarly, the alternative of “predict other questions with similar data” shows that a seizure probability after 1 month can be predicted with an accuracy of 69% without changing the data type to be input.

As described above, the alternative presents a target period from which a better accuracy is expected and a target from which the better accuracy is expected. The data type that is expected to have better accuracy may be presented. The invention is not limited to better accuracy, and the alternative that improves other indicators such as fairness may be presented.

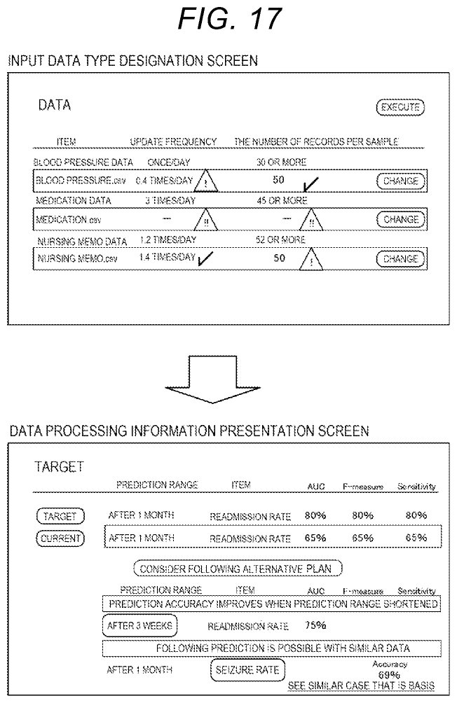

The input data type designation screen of FIG. 17 is the same as in FIG. 16. On the data processing information presentation screen of FIG. 17, area-under-the-curve (AUC), F-measure, and Sensitivity are displayed instead of the accuracy of prediction, and Accuracy indicating the accuracy in the alternative.

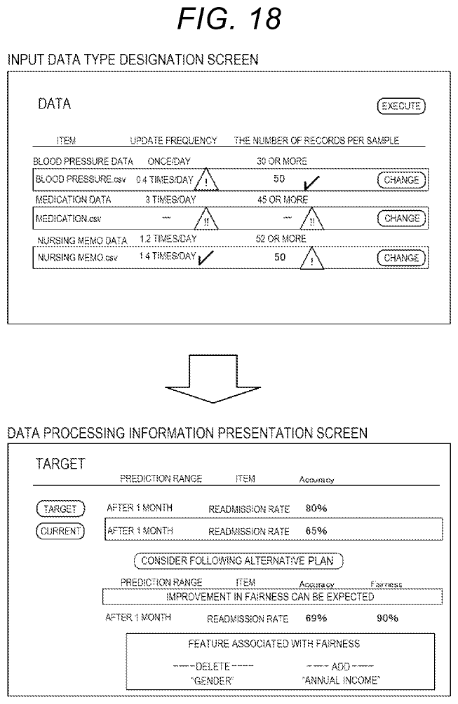

The input data type designation screen of FIG. 18 is the same as that of FIG. 16. On the data processing information presentation screen of FIG. 18, instead of the accuracy prediction, Accuracy is displayed, and in the alternative, Fairness that indicates fairness is displayed, and the addition and deletion of a feature that is effective in improving fairness is presented.

As described above, a data processing assistant system according to the present embodiment includes: a processing record accumulation unit configured to accumulate processing records in which one or more pieces of data, data processing performed using the data, and a processing result of the data processing are associated with each other; a correspondence relation data creation unit configured to create, based on the processing records, correspondence relation data indicative of a correspondence relation among a data type indicating a type of the data, a question to be solved by the data processing, and the processing result; and a processing information presentation unit configured to present, upon receiving designation of the data type and the question, information related to appropriate data processing based on the correspondence relation data. Therefore, the data processing can be assisted by providing a variety of information pertaining to the data processing.

Here, the correspondence relation data may have a hierarchical structure including a question layer having a node indicating the question, a data type layer having a node indicating the data type, and a processing record layer having a node indicating the processing record.

The node may be connected to a single upper node when connected to an upper node located in a relatively upper layer, and may be connected to one or more lower nodes when connected to a lower node located in a relatively lower layer.

The correspondence relation data may further include a classification layer indicating a classification to which a question belongs, the classification layer may be provided above the question layer, the data type layer may be provided below the question layer, and the processing record layer may be provided below the data type layer. The correspondence relation data may include a plurality of the question layers, and a lower question layer may indicate details of the upper question layer. The data type layer of the correspondence relation data may have an individual node for a combination of a plurality of data types.

When the processing information presentation unit traces the hierarchical structure from an upper level based on the designated data type and the designated question and reaches a node connected to the processing record layer, the processing information presentation unit may present data processing related to a processing record connected to the node and/or accuracy of an answer by the data processing.

The processing information presentation unit may calculate similarity between the designated data type and the designated question and a route by which the hierarchical structure is traced from an upper level, and may present data processing related to a processing record connected to a route having strong similarity and/or accuracy of an answer by the data processing.

The data processing assistant system may further include a question searching unit configured to select a node having a high matching level from the node in the data type layer and output a node in the question layer on a route to the node as an answerable question candidate, and the processing information presentation unit may present information related to the appropriate data processing using the designated data type and the question candidate.

The data processing assistant system may further include a necessary data type searching unit configured to, when receiving the designation of the question, trace the hierarchical structure from an upper level based on the designated question and output a node in the data type layer located at a lower level than a node where the necessary data type searching unit reaches as a necessary data type, and the processing information presentation unit may present information related to the appropriate data processing by using the designated question and the necessary data type.

The data processing may work on the one or more pieces of data, generate a feature from the processed data, input the feature to a machine learning model, and set output of the machine learning model as the processing result.

A data processing assistant method according to the present embodiment can provide various information pertaining to data processing by executing: a processing record accumulation step of accumulating processing records in which one or more pieces of data, data processing performed using the data, and a processing result of the data processing are associated with each other; a correspondence relation data creation step of creating, based on the processing records, correspondence relation data indicative of the correspondence relation among the data type indicating the type of the data, the question to be solved by the data processing, and the processing result; and a processing information presentation step of presenting, upon receiving designation of the data type and the question, information related to appropriate data processing based on the correspondence relation data.

The data processing assistant program according to the present embodiment can provide various information related to the data processing by executing following procedures with a computer: a processing record accumulation procedure of accumulating processing records in which one or more pieces of data, data processing performed using the data, and the processing result of the data processing are associated with each other; a correspondence relation data creation procedure of creating, based on the processing records, correspondence relation data indicative of the correspondence relation among the data type indicating the type of the data, the question to be solved by the data processing, and the processing result; and a processing information presentation procedure of presenting, upon receiving designation of the data type and the question, information related to appropriate data processing based on the correspondence relation data.

The above embodiment describes that when the hierarchical structure is traced from the upper level based on the designated data type and the designated question until the node connected to the processing record layer (a node at the lowest input layer), the data processing related to the processing record connected to the node is set as the adaptation. When there is a plurality of data processing as the adaptation, one data processing may be selected by a predetermined index (for example, precision).

Although the description is omitted in the embodiment, when a data type is added or the purpose is changed according to the presented alternative, the processing information presentation unit 22 performs the processing again. When the data type is specified as the starting point, it is possible to add additional information such as the target accuracy, and such additional information can be used for the alternative selection and the like.

The invention is not limited to the above embodiment, and includes various modifications. For example, the above embodiment is described in detail for easy understanding of the invention, and the invention is not necessarily limited to those including all of the configurations described above. The configuration is not limited to being deleted, and may also be replaced or added.

### REFERENCE SIGNS LIST

10: server
11: CPU
12: memory
21: correspondence structure creation unit
22: processing information presentation unit
23: question searching unit
24: necessary data type searching unit
25: screen input and output unit
30: main DB
31: feature set
32: model binary
33: test data
34: prescription record
40: meta DB
41: data processing management data
42: correspondence structure data
43: adaptation table
44: alternative table

What is claimed is:

1. A data processing assistant system comprising:
a processing record accumulation unit configured to accumulate processing records in which one or more pieces of data, data processing performed using the data, and a processing result of the data processing are associated with each other;
a correspondence relation data creation unit configured to create, based on the processing records, correspondence relation data indicative of a correspondence relation among a data type indicating a type of the data, a question to be solved by the data processing, and the processing result; and
a processing information presentation unit configured to present, upon receiving designation of the data type and the question, information related to appropriate data processing based on the correspondence relation data.

2. The data processing assistant system according to claim 1, wherein
the correspondence relation data has a hierarchical structure including a question layer having a node indicating the question, a data type layer having a node indicating the data type, and a processing record layer having a node indicating the processing record.

3. The data processing assistant system according to claim 2, wherein
the node is connected to a single upper node when connected to an upper node located in a relatively upper layer, and is connected to one or more lower nodes when connected to a lower node located in a relatively lower layer.

4. The data processing assistant system according to claim 2, wherein
the correspondence relation data further includes a classification layer indicating a classification to which a question belongs, the classification layer is provided above the question layer, the data type layer is provided below the question layer, and the processing record layer is provided below the data type layer.

5. The data processing assistant system according to claim 2, wherein
the correspondence relation data includes a plurality of the question layers, and a lower question layer indicates details of an upper question layer.

6. The data processing assistant system according to claim 2, wherein
the data type layer of the correspondence relation data has an individual node for a combination of a plurality of data types.

7. The data processing assistant system according to claim 2, wherein
when the processing information presentation unit traces the hierarchical structure from an upper level based on the designated data type and the designated question and reaches a node connected to the processing record layer, the processing information presentation unit presents data processing related to a processing record connected to the node and/or accuracy of an answer by the data processing.

8. The data processing assistant system according to claim 2, wherein
the processing information presentation unit calculates similarity between the designated data type and the designated question and a route by which the hierarchical structure is traced from an upper level, and presents data processing related to a processing record connected to a route having strong similarity and/or accuracy of an answer by the data processing.

9. The data processing assistant system according to claim 2, further comprising:
a question searching unit configured to select a node having a high matching level from the node in the data type layer and output a node in the question layer on a route to the node as an answerable question candidate, wherein
the processing information presentation unit presents information related to the appropriate data processing using the designated data type and the question candidate.

10. The data processing assistant system according to claim 2, further comprising:
a necessary data type searching unit configured to, when receiving the designation of the question, trace the hierarchical structure from an upper level based on the designated question and output a node in the data type layer located at a lower level than a node where the necessary data type searching unit reaches as a necessary data type, wherein
the processing information presentation unit presents information related to the appropriate data processing by using the designated question and the necessary data type.

11. The data processing assistant system according to claim 1, wherein
the data processing works on the one or more pieces of data, generates a feature from the processed data, inputs the feature to a machine learning model, and sets output of the machine learning model as the processing result.

12. A data processing assistant method comprising:
a processing record accumulation step of accumulating processing records in which one or more pieces of data, data processing performed using the data, and a processing result of the data processing are associated with each other;
a correspondence relation data creation step of creating, based on the processing records, correspondence relation data indicative of a correspondence relation among a data type indicating a type of the data, a question to be solved by the data processing, and the processing result; and
a processing information presentation step of presenting, upon receiving designation of the data type and the question, information related to appropriate data processing based on the correspondence relation data.

13. A data processing assistant program that causes a computer to execute:
a processing record accumulation procedure of accumulating processing records in which one or more pieces of data, data processing performed using the data, and a processing result of the data processing are associated with each other;
a correspondence relation data creation procedure of creating, based on the processing records, correspondence relation data indicative of a correspondence relation among a data type indicating a type of the data, a question to be solved by the data processing, and the processing result; and
a processing information presentation procedure of presenting, upon receiving designation of the data type and the question, information related to appropriate data processing based on the correspondence relation data.
